(19) United States
(12) Patent Application Publication
(10) Pub. No.: US 2005/0096481 A1
(43) Pub. Date: May 5, 2005

(54) Method for producing a hydroxyacid esters

(76) Inventors: Rainer Hildebrandt, Scheideholzstieg (DE); Hans-Jurgen Vollmer, Hamburg (DE); Arnold Alscher, Hamburg (DE); Wilhelm Holtmann, Brenneick (DE)

Correspondence Address: THE DOW CHEMICAL COMPANY;INTELLECTUAL PROPERTY SECTION, P. O. BOX 1967, MIDLAND, MI, 48641-1967, US

(21) Appl. No.: 10/489,293

(22) PCT Filed: Oct. 4, 2002
(86) PCT No.: PCT/EP02/11120

(30) Foreign Application Priority Data
Oct. 18, 2001 (AU) PR 8354
Nov. 30, 2001 (AU) PR 9225

(51) Int. Cl. C07C 69/76; C07C 69/66

(52) U.S. Cl. 560/55; 560/179

(57) ABSTRACT

The invention concerns a method for producing esters of a hydroxy acid and a C₁-C₈ alcohol. The invention is characterized by the fact that the esterification is carried out by reactive distillation on a heterogeneous catalyst. The invention makes it possible in particular to produce high purity lactic acid esters in high yields.

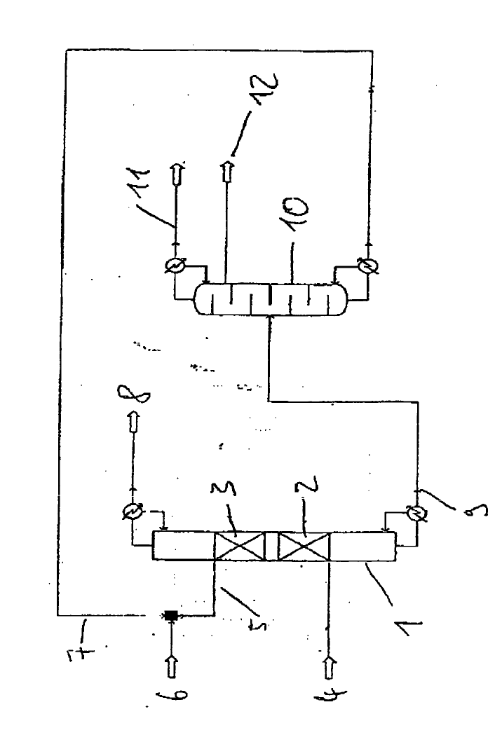

The invention concerns a process for the production of esters of a hydroxy acid and a C₁-C₈ alcohol, especially for the production of lactates.

Lactic acid esters (in the following also called lactates) are suitable solvents for cellulose nitrates, acetates and ethers, chlorine rubber, polyvinyl compounds and the like. They are also used as softeners for cellulose and vinyl resins, as solvents for enamels and as solvents during chip production.

The synthesis of lactic acid esters is usually accomplished by using lactic acid and a corresponding alcohol in the liquid phase in the presence of acid catalysts. Since liquid acid catalysts remain in the final product, the result is a large number of undesired secondary reactions.

The invention has the objective of devising a process such as that described initially which permits the above-noted esters to be produced in a good yield and with high purity.

The invention solves this problem by conducting the esterification by reactive distillation on a heterogeneous catalyst.

The term “reactive distillation” refers to the combination of a chemical reaction (here esterification) and the separation of substances by distillation. It is decisive that immediately following the catalyzed reaction a distillative separation of the products and any remaining educts of the reaction be carried out. As opposed to the case of homogeneous catalysis with a liquid catalyst, in this way the reaction product is immediately separated from the heterogeneous catalyst after the reaction.

A suitable catalyst is any material capable of catalyzing the esterification reaction. Acid catalysts are preferably used. The catalyst is heterogeneous. This means that it is present in a different aggregate state than the educts and products of the esterifications reaction. As a rule a solid-phase catalyst is used.

An α-hydroxy acid, especially preferably lactic acid, is used as the hydroxy acid, An ester is preferably produced with a C₂-C₈ alcohol, especially preferably with a C₂-C₄ alcohol. The alcohol is preferably a primary or secondary alcohol which may be selected from the group consisting of ethanol, n-propanol, isopropanol, n-butanol, 2-butanol and sec-butanol.

The catalyst is preferably fixed in the distillation or rectification column. Packed columns are preferred within the scope of the invention (for a definition, see Ullmann's Encyclopedia Industrial Chemistry, 5th edition, volume B3, p. 4-71). The entire column packing or part of it may be provided with heterogeneous catalyst or replaced by it.

As a support for the fixation of the catalyst a structured packing affixed inside the column may be used such as the packing Katapak®-S by Sulzer Chemtech AG. This packing consists of a wire mesh fabric arranged in layers, and the catalyst can be stored in pockets in the fabric layers and thereby fixed in place.

As catalysts preferably one will use acid ion exchange resins such as the macroporous acid ion exchange resins called Amberly®15 by Rohm & Haas. Commercially available pellets of this catalyst with a diameter between 0.35 at 1.2 mm may be used.

The desired contact time of the educts in the column may be controlled by variation of the distillation/rectification parameters. The higher-boiling educt (usually lactic acid) is preferably fed into the distillation column above the catalyst, the lower-boiling educt below the catalyst. The educts in this way are brought to reaction with each other in the region of the catalyst packing in cocurrent. The reaction parameters may also be influenced by the choice of the reflux ratio of the column, the column temperature and (depending on it) the pressure in the column.

A special advantage of reactive distillation is the fact that during the esterification the water of reaction which forms is immediately removed by distillation, and therefore the reaction equilibrium is shifted in the direction of the ester. In addition, in this way any water possibly contained in the educts is removed. Industrial lactic acid, for example, usually has a water content of about 20%.

When necessary in order to remove the water additionally a suitable entraining agent may be used with which the water present in the educts as well as the water of reaction is distilled off azeotropically. Under some conditions an alcohol used for esterification may even serve as the entraining agent. During the production of isopropyl ether, the isopropanol used in excess may serve as simultaneously as a water entraining agent.

The reaction temperature on the catalyst may range around 60-200° C., preferably 60-150° C., even more preferably 70-100° C. This temperature depends on the boiling points of the liquid and gaseous products being distilled in the column which may be varied if necessary by adjusting either an excess pressure or a low pressure in the column. An upper limit to the temperature is also determined by the catalyst material used. For example, styrene-based catalysts are temperature-stable only up to about 95-100° C., at higher temperatures they split off sulfuric acid. Silicone-based catalysts are frequently more temperature-stable up to, e.g., about 200° C. The pressure in the column is generally selected in such a way that the alcohol used boils at a temperature which lies below any potential decomposition temperature of the heterogeneous catalyst.

The low-volatility ester obtained in the reaction is drawn off from the column as a bottom product. In the case of high-boiling esters the sump temperature may sometimes be so high that decomposition reactions occur. In this case to lower the sump temperature a lower-boiling solvent can be cut into the sump which can be removed in a subsequent distillation step.

The use of a heterogeneous catalyst according to the invention within the scope of reactive distillation has the additional advantage that the metal ion impurities contained in the educts are removed from the liquid phase as the catalyst is deactivated. In this way one obtains highly pure esters which may be used especially advantageously as solvents for photo enamels or the like during chip production.

The invention is described in the following with reference to examples of embodiment. The figure shows schematically an installation for realization of the process according to the invention.

A packed rectification column 1 displays several sections. Two sections with the reference numbers 2 and 3 display a catalytic packing which may involve the above-mentioned Katapak®-S by Sulzer Chemtech AG. This catalytic packing is filled with an acid ion exchange resin in the form of pellets.

Alcohol is fed into the column 1 at 4, at 5 lactic acid introduced at 6 is fed in together with lactic acid dimer which is returned through conduit 7, coming from the below-described refining of the crude ester. At the head of the column at 8 water (reaction water and possibly water contained in the initial products) is drawn off from the packed column 1 possibly with the aid of an entraining agent.

The crude ester is drawn off from the column sump through conduit 9. It typically displays a purity of 90-95%. This crude ester is subjected to refining by distillation in a conventional column 10. At the head of this column 10 at 11 the remaining high-volatility components such as the residual water and alcohol are drawn off. The lactic acid ester is taken off from the column at 12. Relative to the lactic acid used, it is obtained in a yield which usually lies above 95%. The sump of this distillative refining system essentially contains the dimeric lactic acid formed as a secondary product, which is sent through the above-noted conduit 7 back to the packed rectification column 1 as an educt. This problem-free reusability of dimeric lactic acid is a particular advantage of the present invention. In the state-of-the-art with liquid catalysts the reaction mixture has to be neutralized in order to suppress other reactions. This neutralization makes a subsequent immediate reutilization (recyclability) of the refined secondary products impossible.

All percentage data in the examples are percent by weight unless otherwise stated.

### EXAMPLE 1

### Preparation of N-propyl Lactate

| | |
|---|---|
| Installation | A continuous distillation system with 5 sections. Lowest section and |
| | section 4 and 5 with 40 mm diameter with Sulzer CY ® packing, |
| | sections 2 and 3 with 70 mm diameter and catalytic packing. Phase |
| | separator at head with return of organic phase through section 4. Lactic |
| | acid is input through section 3 and propanol through section 1 |
| Entraining agent: | 445 g n-heptane first filling, further addition as needed as head |
| | temperature rises |
| Reflux | 1:0.2 |
| Inputs | |
| n-propanol | Through first bottom with 90° C., raised up to 230 g/h, total 13448 g |
| Lactic acid | Through third bottom at 90° C., raised up to 350 g/h, total 15850 g |
| Cycles: | |
| Water phase: | 150-170 g/h, total 10 308 g |
| Sump: | 420-480 g/h, total 18 to 88 g, residual sump 1350 g/h |
| Temperatures: | |
| Head: | 69-70° C. |
| Through 3rd ring* | 74-75° C. |
| Through 2nd ring: | 91-92° C. |
| Through 1st ring: | 92-93° C. |
| Sump | Up to 165° C., later reduced to 144-146° C. |
| Water content of | 82-85% |
| water phase | |
| Acidity in the sump: | 0.40-0.81 mg KOH/g |

*German: Schuβ = ring, collar

Katapak® filled with an acid ion exchange resin was used as the catalytic packing

n-Heptane was used a the agent for entraining the water of reaction and the water contained in the initial products. The lactic acid used was 80% with a water content of 20%. The column was filled with n-propanol and heptane before startup. The completeness of the reaction relative to the sump product was determined from the acidity in the sump. The feed volumes (inputs of n-propanol and lactic acid) were adjusted such that this acidity in the sump was clearly below 1 mg KOH/g.

The sump temperature was initially stabilized at about 165° C.; the n-propanol content in the sump was less than 1%. This high sump temperature promotes further and secondary reactions of the sump products; therefore a little n-propanol (5-7%) was sliced into the sump and the sump temperature thus lowered to about 144-146° C. The content of n-propyl lactate in the sump was determined by gas chromatography and amounted to approximately 93.5%.

The sumps of the reactive distillation were distilled in a packed column. The following fractions were obtained:
1. 3.1% first run with 99.5% n-propanol.
2. 0.3% intermediate run with 25% n-propanol and 75% propyl lactate
3. 89.3% propyl lactate with a purity of 99.75%. The impurities were 0.13% n-propanol and 0.02% water. The acidity was 0.08 mg KOH/g.
4. 70% residual sump with an acidity of 7.1 mg KOH/g. The sump residue contained predominantly dimers and oligomers.

The first run, intermediate run and sump residue were reutilized in the reactor distillation without problem.

The finished product was studied gas chromatographically and displayed a content of n-propyl lactate of99.77%. The n-propanol content was 0.13%, the water content 0.02% and the acidity 0.08 mg KOH/g.

### EXAMPLE 2

### Production of Ethyl Lactate

| | |
|---|---|
| Installation | A continuous distillation system with 5 sections. Lowest section and |
| | sections 4 and 5 with 40 mm diameter with Sulzer CY ® packing, |
| | sections 2 and 3 with 70 mm diameter and catalytic packing, Phase |
| | separator at head with return of organic phase through section 4. |
| | Lactic acid input through section 3 and ethanol through section 1 |
| Entraining agent: | 120 g diisopropyl ether first filling, further addition as needed as head |
| | temperature rises. Later also addition of cyclohexane as entraining |
| | agent |
| Reflux | 1:0.5 |
| Inputs | |
| n-propanol | Through first bottom with 80° C., ca. 30 g/h, total 3148 g |
| Lactic acid | Through third bottom at 90° C., ca. 50 g/h, total 2631 g |
| Cycles: | |
| Water phase: | 35 g/h, total 1725 g, water content 54-63% |
| Sump: | 55 g/h, total 2269 g, water content 0.1-0.2%, acidity 5-9 mg KOH/g |
| Temperatures: | |
| Head: | 61° C. |
| through 4th ring: | 62° C. |
| through 3rd ring: | 68-70° C. |
| through 2nd ring: | 78-79° C. |
| through 1st ring | 79° C. |
| Sump | 102-130° C. depending on residual alcohol content |
| Water content of | 54-63% |
| water phase | |
| Acidity in the sump: | 5-9 mg KOH/g |

Before startup the column was filled with ethanol and diisopropyl ether as entraining agents. The acidity in the sump was somewhat higher than in the case of synthesis of n-propyl lactate, because at the low temperatures in the column the esterifications reaction took place only relatively slowly. The reactive distillation under excess pressure with an accordingly elevated column temperature may accelerate the rate of the reaction.

At a sump temperature of about 130° C. the ethyl lactate content in the sump amounted to 80.7% and the ethanol content 5.7%. The dilactide content was 3%.

During the distillation in a packed column the following quantities were obtained:
1. 16.6% first run—ethanol with 12.5% water
2. 2.9% intermediate run—ethanol with 34% water
3. 18.8% ethyl lactate with a purity of 99.6% and 0.4% water
4. 42.5% ethyl lactate with 1.2% water
5. 18.8% sump residue you with an acidity and 22 6 mg KOH/g. The sump residue contained predominantly lactic acid and dimer.

Fractions 3 and 4 could be redistilled without decomposition. The redistillation yielded ethyl lactate with low acidity and high purity.

The residue of the refining process contained high contents of lactic acid and dilactide and can be fed back to the reactive distillation column ad educt. Mixing this residue with about 10% water is advantageous for facilitating the splitting the dilactide back into lactic acid.

When the accumulated residues were reutilized in the manner described the total yield of ethyl lactate relative to the lactic acid used was more than 95%.

### EXAMPLE 3

### Production of Isopropyl Lactate

| Test series | 1 | 2 |
|---|---|---|
| | 2 catalytic sections | 3 catalytic sections |
| Installation: | Continuous distillation system with five or six | |
| | sections. Bottom section and sections 5 6 with | |
| | 40 mm diameter with Sulzer see why packing, | |
| | sections 2 and 3 (test series 1) and sections | |
| | 2, 3 and 4 (test series 2) with 70 mm diameter and | |
| | catalytic packing. Lactic acid input through | |
| | catalytic sections and ethanol through section 1 | |
| Pressure: | Standard pressure | Standard pressure |
| Differential pressure: | 2.5 mbar | 3 mbar |
| Reflux | 1:7 to 1:8.5 | 1:7.5 to 1:8 |
| Inputs: | | |
| IPA: | Through 1st plate with | Through 1st plate with |
| | 80° C. | 80° C. |
| | 350 g/h total 8600 g | |
| Lactic acid: | Through 3rd plate with | Through 4th plate with |
| | 95° C. | 95° C. |
| | 75 g/h, total 1850 g | 140 g/h, total 2160 g |
| Cycles | | |
| Head product: | 330 g/h, with 8-12% | 550 g/h, with 9-10% |
| | water, total 8060 g | water, total 8010 g |
| Sump: | 100 g/h total 1002 g, | 180 g/h, total 2560 g, |
| | water content <0.02% | water content <0.02% |
| | Acidity 3-4 mg | Acidity 10-17 mg |
| | KOH/g | KOH/g |
| Temperatures: | | |
| through 6th ring | — | 80° C. |
| through 5th ring | 80° C. | 81° C. |
| through 4th ring | 81° C. | 82° C. |
| through 3rd ring | 81.5° C. | 82° C. |
| through 2nd ring | 82° C. | 82° C. |
| through 1st ring | 82° C. | 83° C. |
| Sump: | 92-152° C. | 125-153° C. |
| | depending on residual | depending on residual |
| | alcohol content | alcohol content |

In test series 1 the column sections 2 and 3 were filled with a catalytic packing, in test series 2 the reactive zone was lengthened by one section. As the entraining agent an isopropanol excess was utilized. The isopropanol/water stream was taken off through the head. The lactic acid introduced above the catalytic sections was 80% (water content 20%). The column was filled with isopropanol before startup.

One recognizes that the lengthening of the reactive zone by one catalytic pacing almost doubled the throughput (140 instead of 75 g/hour lactic acid) at almost the same acidity of the sump. The isopropyl acetate content in the sump was between 76 and 85%. The taken-off head product contained diisopropyl ether formed as a secondary product by etherifications of isopropanol.

The sumps of the reactive distillation were distilled in a packed column. Typically the following quantities were obtained:
1. 16.5% first run—isopropanol 99.8%
2. 1.0% intermediate run—40% isopropanol and 60% isopropyt lactate
3. 64.3% isopropyl lactate with a purity of 99.65%, 0.11% water and an acidity of 0.07 mg KOH/g
4. 15.3% sump residue with circa 50% isopropyl lactate, circa 35% dialect tied and an acidity of
   59 mg KOH/g.

The sump residue 4 was reutilized as educt in the reactive distillation without problem.

1. Process for the production of esters of a hydroxy acid and a C₁-C₈ alcohol, characterized by the fact that the esterification is accomplished by reactive distillation on a heterogeneous catalyst.

2. Process as in claim 1, characterized by the fact that the hydroxy acid is an α-hydroxy acid.

3. Process as in claim 2, characterized by the fact that the α-hydroxy acid is lactic acid.

4. Process as in one of claims 1-3, characterized by the fact that the alcohol is a C₁-C₈ alcohol.

5. Process as in claim 4, characterized by the fact that the alcohol is a C₂-C₄ alcohol.

6. Process as in one of claims 1-5, characterized by the fact that the alcohol is a primary or secondary alcohol.

7. Process as in claim 6, characterized by the fact that the alcohol is chosen from the group consisting of ethanol, n-propanol, isopropanol, n-butanol, 2-butanol and sec-butanol.

8. Process as one of claims 1-7, characterized by the fact that the catalyst is fixed in the distillation column.

9. Process as in claim 8, characterized the fact that the catalyst is an acid ion exchange resin.

10. Process as claims 8 or 9, characterized by the fact that the lower-boiling-initial product is fed into the distillation column below the catalyst.

11. Process as one of claims 8-10, characterized by the fact that the higher-boiling initial product is fed into the distillation column above the catalyst.

12. Process as one of claims 1-11, characterized by the fact that the water contained in the initial products as well as the water of reaction is removed by an entraining agent.

13. Process as one of claims 1-12, characterized by the fact that the reaction temperature is 60-200° C., preferably 60-150° C., even more preferably 70-100° C.

14. Process as one of claims 1-13, characterized by the fact that a solvent is sliced into the sump in order to lower the sump temperature.
